(19) United States
(12) Patent Application Publication
(10) Pub. No.: US 2014/0343406 A1
(43) Pub. Date: Nov. 20, 2014

(54) NEEDLE POSITIONING DEVICE FOR ULTRASOUND-GUIDED TREATMENTS AND EXAMINATIONS

(71) Applicant: LAD technology LLC, Hellertown, PA (US)

(72) Inventor: Dusan Damjanovic, Hellertown, PA (US)

(73) Assignee: LAD technology LLC, Hellertown, PA (US)

(21) Appl. No.: 14/278,201

(22) Filed: May 15, 2014

Related U.S. Application Data
(60) Provisional application No. 61/855,418, filed on May 15, 2013.

(51) Int. Cl. A61B 8/08; A61N 7/00; A61B 8/00

(52) U.S. Cl. CPC A61B 8/0841; A61B 8/4218; A61N 7/00
USPC 600/424

(57) ABSTRACT

A device for positioning a needle in an ultrasound-guided treatment or examination is disclosed. The device may include a main body portion attached to an ultrasound probe, an adjustable arm member attached to the main body member, and a needle mount attached to the arm member. The probe casts an ultrasound beam to identify a target portion of a material. The arm member is adjustable to position a needle attached to the needle mount in a desired position relative to a desired point of entry into the material. The needle mount is adjustable to position the needle at a desired insertion angle into the material. The needle may be inserted into the material to deliver medication in a vicinity of the target portion of the material, or extract fluid or a material sample from the target portion of the material. A system and method employing the device are also disclosed.

### CROSS REFERENCE TO RELATED APPLICATIONS

This application claims the benefit of U.S. Provisional Application No. 61/855,418, filed on May 15, 2013, the entire disclosure of which is incorporated herein by reference for all purposes.

### BACKGROUND

Ultrasound devices are often used to assist health care providers in administering nerve block (local anesthesia) treatments, as well as other treatments and examinations (e.g., biopsies and blood vessel cannulations), to patients in preparation for surgical procedures. For example, according to a conventional procedure for administering a nerve block treatment to a patient, a first treatment provider or user (e.g., anesthesiologist or anesthetist) must hold a probe of the ultrasound device (“ultrasound probe”) in one hand to cast and manipulate a peripheral nerve block needle for delivering the nerve block medication(s) in the other hand. The ultrasound probe casts an ultrasound beam that enables a target nerve to be visually identified, so that the first provider can accurately insert the needle through the patient's body tissue and into a vicinity of the target nerve without damaging the nerve. While the first treatment provider is operating the ultrasound probe and the nerve block needle, a second treatment provider operates an electronic nerve stimulator or other device to verify the location of the target nerve, and operates a syringe to administer the nerve block medication(s) in the vicinity of the target nerve through the needle. The location, speed and pressure of the injection of nerve block medication is critical for patient safety and requires continuous, accurate communication between the two treatment providers.

In-plane nerve block injections, in which the needle is positioned substantially entirely within the plane of the ultrasound beam when the needle is inserted into tissue, are particularly challenging. The needle must be kept in view at all times, which is difficult but important for safety and accuracy of the nerve block. The ultrasound beam is narrow, and it requires a great deal of attention by the first treatment provider, which diminishes the ability of the first treatment provider to attend to the patient and communications from the second treatment provider.

In view of the above, it is desirable to provide improved devices, systems and methods for administering ultrasound-guided medical treatments and examinations. Particularly, it is desirable to provide devices, systems and methods that increase patient safety and are suitable for implementation by a single user/treatment provider. Further still, it is desirable that such devices, systems and methods be cost-effective and simple to implement.

### SUMMARY

The disclosure is directed to a needle positioning device for ultrasound-guided treatments and examinations. The disclosure further concerns an ultrasound system including a needle positioning device, and ultrasound treatment and examination methods using a needle positioning device.

According to an embodiment, a needle positioning device for an ultrasound-guided medical treatment or examination includes a main body member, an arm member and a needle mount. The main body member is configured to attach to an ultrasound probe. The arm member includes a first arm portion attached to the main body member, and a second arm portion that is pivotable with respect to the first arm portion. The needle mount is attached to the second arm portion and is configured to retain a needle and vary an angle of insertion of the needle into a material.

At least one motor may be configured to pivot the needle holder to vary an angle of insertion of the needle, and move the needle linearly with respect to the needle mount.

The main body member may include control elements configured to control operation of the at least one motor.

According to another embodiment, an ultrasound system for performing an ultrasound-guided treatment or examination includes an ultrasound probe configured to cast an ultrasound beam, and a needle positioning device. The needle positioning device includes a main body member attached to the ultrasound probe, an arm member attached to the main body member and a needle attached to the arm member. The arm member is adjustable to position a needle in a desired position relative to a point of entry into a material. The needle mount is attached to the arm member, and is configured to retain a needle and vary an angle of insertion of the needle into a material.

At least one motor may be configured to pivot the needle holder to vary an angle of insertion of the needle, and move the needle linearly with respect to the needle mount.

The main body member may include control elements configured to control operation of the at least one motor.

According to another embodiment, an ultrasound treatment method is provided. According to the method, an ultrasound probe is operated to cast an ultrasound beam through a material. Based on the ultrasound beam, an arm member of a needle positioning device attached to the ultrasound probe is adjusted to position a needle in a desired position relative to a point of entry into the material. A needle mount attached to the arm member is adjusted to determine an angle of insertion of the needle into the material, and the needle is inserted into the material.

The arm member may include a first arm portion and a second arm portion pivotally connected to the first arm portion, and the arm member may be adjusted by pivoting the second arm portion with respect to the first arm portion.

At least one motor may be operated to adjust the needle mount and insert the needle into the material. Adjusting the needle mount may include pivoting the needle mount with respect to the arm member. Inserting the needle into the material may include moving the needle with respect to the needle mount

Other features and advantages are apparent from the following detailed description, the drawings and the claims.

### BRIEF DESCRIPTION OF THE DRAWINGS

FIG. 1 shows a system including an ultrasound device and an exemplary needle positioning device connected to an ultrasound probe of the ultrasound device, wherein the needle positioning device is in a first configuration for an in-plane nerve block treatment, according to an embodiment of the invention.

FIG. 2A is a perspective view of a main body member of the needle positioning device, showing the main body member in an open, unlocked configuration.

FIG. 2B is a front end view of the main body member.

FIG. 2C is a first side view of the main body member.

FIG. 2D is a second side view of the main body member, opposite the first side view.

FIG. 2E is a top view of the main body member in a closed, locked configuration for securing the needle positioning device to the ultrasound probe.

FIG. 3A is a front view of an arm member and a needle mount of the needle positioning device.

FIG. 3B is a top view of the arm member and needle mount.

FIG. 3C is a first, proximal end view of the arm member and needle mount.

FIG. 3D is a second, distal end view of the arm member and needle mount.

FIG. 4 is a front perspective view of a needle assembly of the needle positioning device.

FIG. 5 shows the needle positioning device in a second configuration for an out-of-plane nerve block treatment.

### DETAILED DESCRIPTION

The following detailed description concerns exemplary devices for positioning a needle in an ultrasound-guided medical treatment or examination, and ultrasound systems including such devices. The description further concerns methods of administering ultrasound-guided medical treatments and examinations using needle positioning devices.

The following detailed description is provided to assist the reader in gaining a comprehensive understanding of the devices, systems, and/or methods described herein. Accordingly, various changes, modifications, and equivalents of the devices, systems and/or methods described herein will be suggested to those of ordinary skill in the art. Also, descriptions of well-known functions and constructions may be omitted for increased clarity and conciseness.

Various directions and orientations (e.g., “up”, “down”, “horizontally”, “vertically”, “front”, “rear”, “side”, “top”, “bottom”, “proximal” and “distal”) are referenced throughout the detailed description. The directions and orientations referenced in the detailed description are provided to facilitate understanding of the exemplary embodiments disclosed herein, and are not intended to be limiting.

Reference numerals and/or characters repeated throughout the detailed description and appended drawings indicate similar components and features.

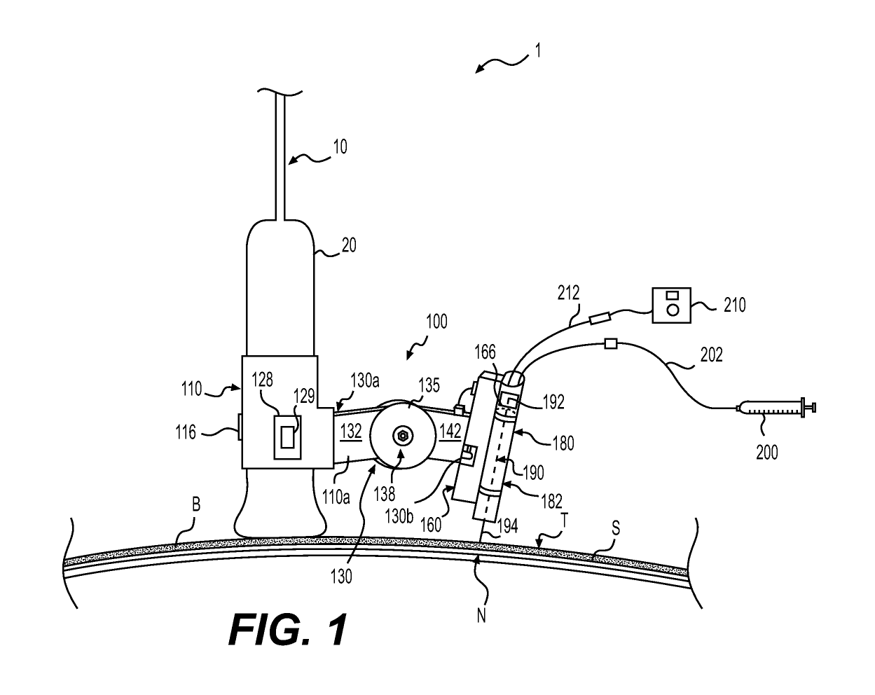

FIG. 1 shows an exemplary ultrasound system 1 for performing an ultrasound-guided treatment. The system 1 includes an ultrasound device 10 and a needle positioning device 100 configured to operate in conjunction with the ultrasound device 10. In FIG. 1, the needle positioning device 100 is shown in a first configuration for delivering an in-plane nerve block treatment.

As shown in FIG. 1, the ultrasound device 10 includes an ultrasound probe 20 that is operable to cast an ultrasound beam B through tissue T (e.g., skin, muscle, etc.) of a patient (e.g., person or animal). The ultrasound beam B may enable a treatment provider/operator of the system 1 to visually locate a target nerve N within the tissue T so that a nerve block medication (e.g., local anesthetic) may be administered to a vicinity of the nerve N or a nerve sheath S encapsulating the nerve N.

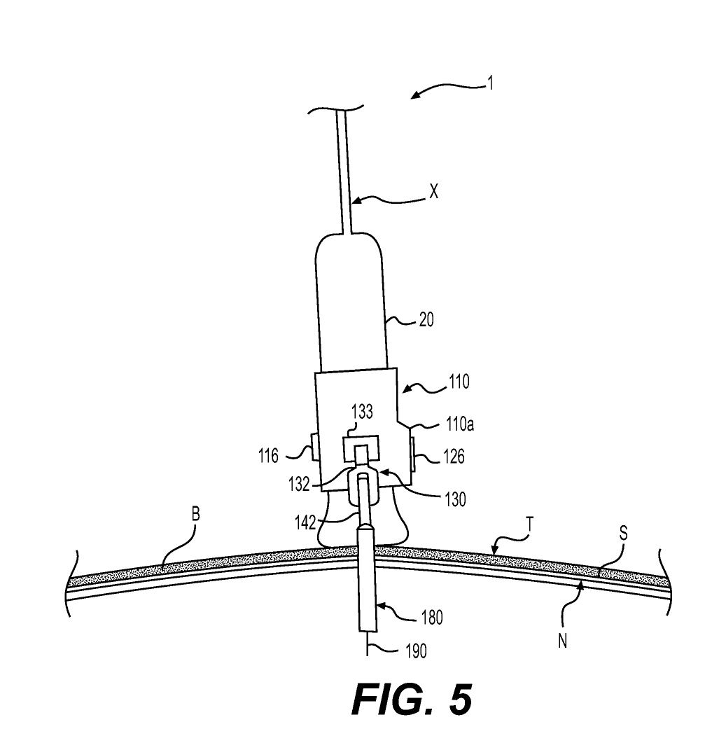

Still referring to FIG. 1, the needle positioning device 100 includes a main body member 110, an adjustable arm member 130 having a proximal end 130a attached to a front end 110a of the main body member 110, and a needle mount 160 attached to a distal end 130b of the main body member. A needle assembly 180 including a needle holder or needle cartridge 182 and a needle 190 for delivering nerve block medication to the vicinity of the target nerve N is attached to the needle mount 160. Since the device 100 as shown in FIG. 1 is configured for an in-plane nerve block treatment, the arm member 130 is aligned substantially parallel to the plane of the ultrasound beam B, such that the needle 190 will lie substantially entirely within the plane of the ultrasound beam B when the needle 190 is inserted into the tissue T. In an alternate embodiment shown in FIG. 5, the device 100 may be configured for an out-of-plane nerve block treatment in which the arm member 130 is aligned substantially perpendicular to the plane of the ultrasound beam B, such that the needle 190 will be positioned transverse to the plane of the ultrasound beam B when the needle 190 is inserted into the tissue T. As described below in greater detail, the main body member 110 is configured to secure the device 100 to the ultrasound probe 20, the arm member 130 is adjustable to position the needle 190 at a desired position relative to (e.g., on top of) a desired point of entry into the tissue T, and the needle mount 160 is adjustable to determine an angle at which the needle 190 is advanced into the tissue T.

A syringe 200 storing the nerve block medication may be connected to the needle 190 through a fluid delivery line 202 for supplying the nerve block medication to the needle 190. Additionally, a nerve stimulation device 210 may be electrically connected to the needle 190 by a conductive wire 212 for delivering electrical signals to the nerve N in order to verify the location of the nerve N.

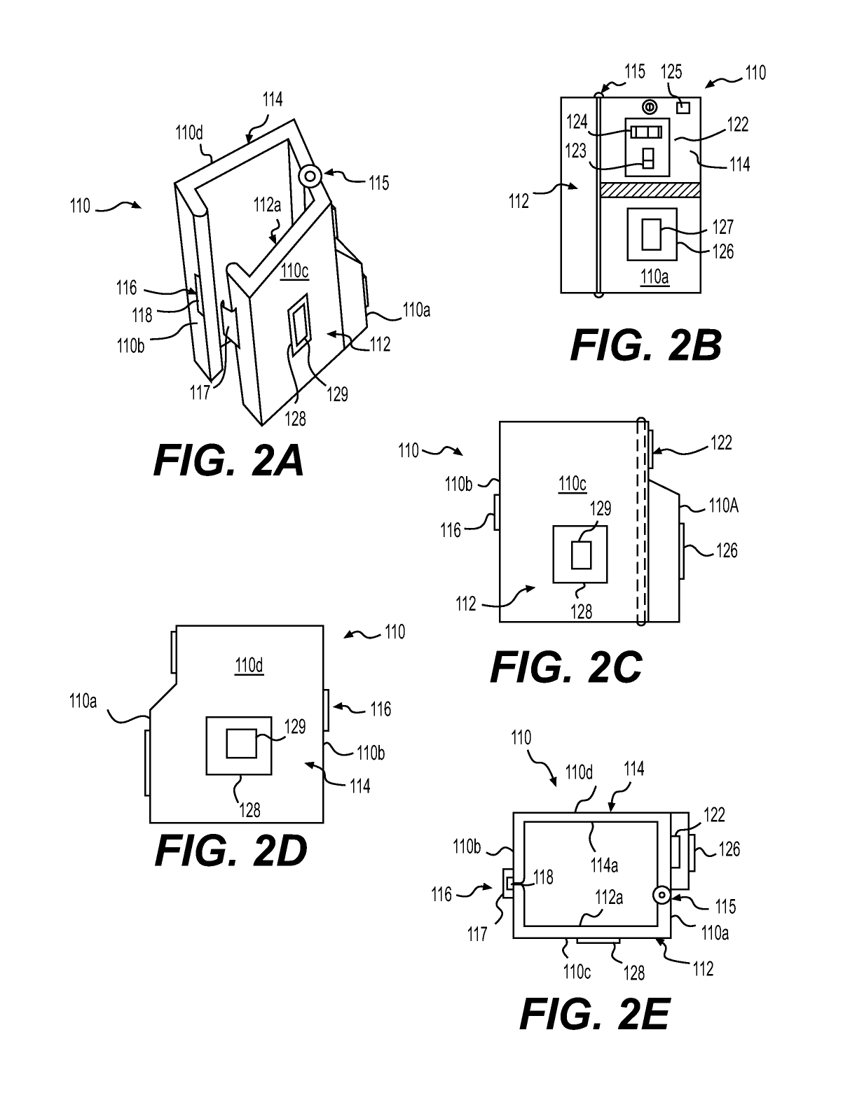

The main body member 110 is shown in detail in FIGS. 2A-2E. The main body member 110 may be configured to clamp onto an outer surface of the ultrasound probe 20 in order to secure the device 100 to the probe 20. Referring to FIG. 2A, the main body member 110 includes a first body portion 112 and a second body portion 114 that may be pivotally connected to each other by a pivot connection or hinge connection 115. The first and second body portions 112, 114 may have, for example, substantially C-shaped cross-sections, and may be arranged in opposing orientations as shown in FIGS. 2A and 2E. Preferably, the inner surfaces 112a, 114a are shaped to tightly engage the outer surface of the probe 20 when secured onto the probe 20. As ultrasound probes may vary in size and/or shape, other shapes and configurations are possible for the body portions 112, 114 to enable attachment to different types of ultrasound probes. Additionally, interchangeable resilient sleeve members (not shown) of various thicknesses and/or shapes may be provided for attachment to the inner surfaces 112a, 114a of the body portions 112, 114. The sleeve members may be designed to conform to the sizes and shapes of the outer surfaces of certain probes, thereby enabling the main body member 110 to tightly attach to probes of various outer dimensions and configurations.

As shown in FIGS. 2A-2E, the main body member 110 has a front end 110a, a rear end 210b, a first side 110c extending between the front and rear ends 110a, 110b, and a second side 110d extending between the front and rear ends 110a, 110b, generally opposite the first side 110c. Referring to FIGS. 2A and 2E, a closure mechanism 116 is provided on the rear end 110b for fastening the body portions 112, 114 together to place the main body member 110 in a closed configuration surrounding the ultrasound probe 20, and thereby firmly attach the main body member 110 to the probe 20. The closure mechanism 116 may include a first locking member 117 attached to the first body portion 112 and a second locking member 118 attached to the second body portion 114. The locking members 117, 118 may be configured to engage each other in an interlocking fashion, and may include any combination of projections and recesses, or other components configured to interlock with each other and thereby lock the first and second body portions 112, 114 together to form the closed configuration of FIG. 2E. The locking members 117, 118 may be disengaged and the body portions 112, 114 may be pivoted apart to place the main body member 110 in the open configuration of FIG. 2A in order to allow the device 110 to be detached from the probe

FIG. 2B provides a detailed view of the front end 110a of the main body member 110. As shown in FIG. 2B, an on/off power button 120, a control panel 122 and an in-plane arm member connection port 126 may be located on the front end 120a. The control panel 122 may include a first control element 123 (e.g., button(s) or joystick(s)) configured to control vertical movement of the needle 190 and a second control element 124 (e.g., button(s) or joystick(s)) configured to control lateral displacement of the head of the needle 190 (and thus, an insertion angle of the needle 190 into the tissue T). The in-plane arm member connection port 126 is a female-type port including a receptacle 127 provided with one or more electrical contacts for making an electrical connection with the arm member 130. The power button 120, control panel 122 and the electrical contact(s) of the receptacle 127 are electrically connected to a DC or AC power source (not shown), such as batteries or an AC electrical outlet, through electrical circuitry (not shown) provided in the main body member 110. The electrical receptacle 127 is also electrically connected to the control elements 123, 124.

An external control input port 125 may also be included in the main body member 110. An external control device (e.g., handheld unit and/foot pedal(s)) may be connected to the input port 125 to supplement or replace the control elements 123, 124.

As shown in FIG. 2C, a first out-of-plane arm member connection port 128 is provided on the first side 110c of the main body member 110 for connecting the arm member 130 to the main body member 110 in a configuration for an out-of-plane nerve block treatment (FIG. 5). Additionally, as shown in FIG. 2D, a second out-of-plane arm member connection port 128 may be provided on the second side 110d of the main body member 110. The out-of-plane arm member connection ports 128 are female-type ports including receptacles 129 provided with one or more electrical contacts that are electrically connected to the DC or AC power source and the control elements 123, 124.

It should be understood that, while FIGS. 2A-2E show specific configurations and locations of the closure mechanism 116, power button 120, control panel 122 and connection ports 126, 128, it should be understood that other configurations and locations are possible. Particularly, different configurations and locations are possible for these components based on ergonomic considerations to facilitate ease of use.

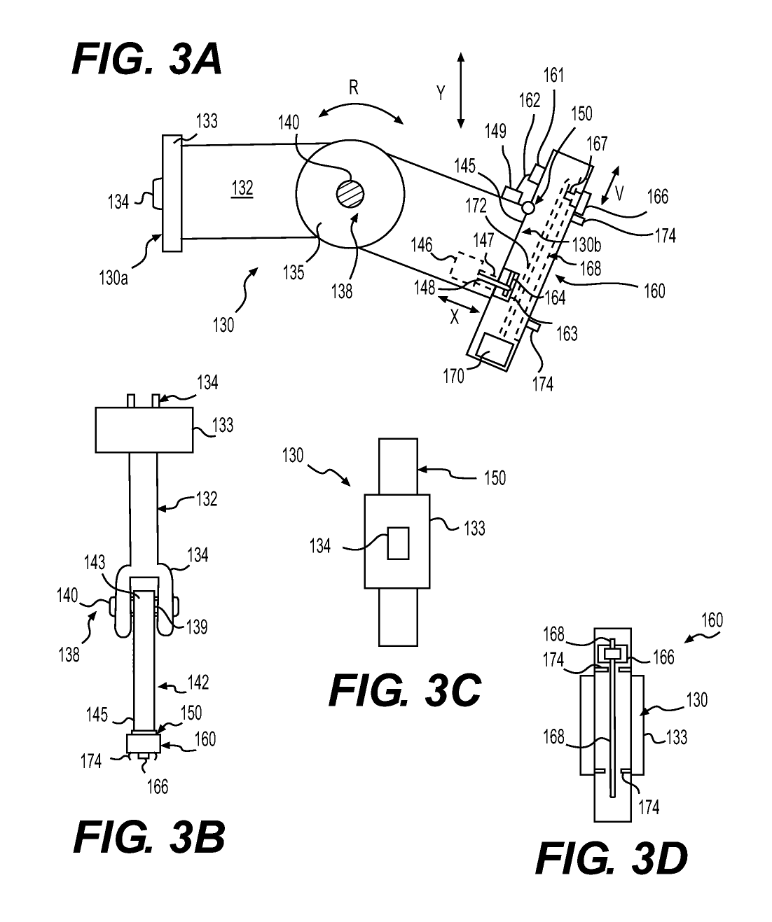

FIGS. 3A-3D show the arm member 130 in detail. As best illustrated in FIGS. 3A and 3B, the arm member 130 includes a first arm portion 132 forming the proximal end 130a of the arm member 130, and a second arm portion 142 forming the distal end 130b of the arm member 130. As shown in FIG. 3C, the first arm portion 132 may include a connector 134 at a first end 133 corresponding to the proximal end 130a of the arm member 130. The connector 134 may be in the form of a post, prong or other male-type projection provided with one or more electrical contacts, and configured to connect to one of the connection ports 126, 128 by insertion into a respective receptacle 127, 129 to form an electrical connection with the AC or DC power source and the control elements 123, 124. The connection between the connector 134 and one of the receptacles 127, 129 also forms a secure mechanical connection between the arm member 130 and the main body member 110. By way of example, the receptacles 127, 129 may include spring-biased detent members (not shown) configured to engage the connector 134 in a locking fashion when the connector is fully inserted in the receptacles 127, 129. While the main body member 110 is shown and described as having connection ports 126, 128 and the first arm portion 132 is shown and described as having a male-type connector 134, the configuration may be reversed such that male-type connectors are provided in the main body member 110 and a female-type connection port is provided in the first arm portion 132. According to additional embodiments, the main body member 110 and arm member 130 may be attached by other known types of interlocking connectors that slide or snap together, instead of the connector 134 and connection ports 126, 128.

Continuing with reference to FIGS. 3A and 3B, the first arm portion 132 may include a second end 135 having a forked configuration, and configured to receive a first end 143 of the second arm portion 142. The second end 135 of the first arm portion 132 and the first end 143 of the second arm portion 142 may be pivotally connected by a pin or rod 139 at a pivot joint 138. A pivot lock/release button or actuator 140 may be provided in the second end 135 of the first arm portion 132, at or near the pivot joint 138. The pivot lock/release actuator 140 may be manually operable to selectively lock and unlock a detent mechanism (not shown) in the pivot joint 138. The detent mechanism may be any suitable known type of detent mechanism. Locking of the detent mechanism prevents pivoting of the arm portions 132, 142 about the pivot joint 138 in order to maintain a desired position of the needle 190 at or near a desired point of entry into the tissue T, while unlocking of the detent mechanism enables the arm portions 132, 142 to pivot (R) about the pivot joint 138 to position the needle 190 in a desired position relative to a desired point of entry into the tissue T. Pivoting of the arm portions 132, 142 displaces the needle mount 160 primarily in vertical directions (Y) with respect to the tissue T.

Referring to FIG. 3A, A needle adjustment motor 146 is provided in the second arm portion 142, and is electrically connected to the connector 134 to receive power from the power source and electrical control signals from the second control element 124 or an external control device connected to port 125 (FIG. 2B). Accordingly, control of operation of the motor 146 using the control element 124 or an external control device is enabled. The motor 146 may be any suitable type of electrical motor. The motor 146 is configured to rotate in first and second directions through operation of the control element 124, thereby enabling turning of the lead screw 148 in two opposing rotational directions. A lead screw 148 may be disposed in a bore hole 147 in a bottom area of the second arm portion 142, and may be connected to the motor 146 such that the lead screw 148 turns in response to rotation of the motor 146.

Also as shown in FIG. 3A, the second arm portion 142 includes an electrical connector 149 that is electrically connected to an electrical connector 161 of the needle mount 160 by a wire 162. Thus, the needle mount 160 is provided with electrical connections to the power source and the control elements 123, 124.

As shown in FIGS. 3A and 3B, the needle mount 160 may be pivotally connected to the distal end 130b of the arm member 130 by a pivot connection 150. The pivot connection 150 may be formed between a top area of the needle mount and a top area of the second end 145 of the second arm portion 142. As illustrated in FIG. 3A, the needle mount 160 may include a recess configured to receive the lead screw 148, and a screw follower 164 configured to engage threads of the lead screw 148, such that turning of the lead screw 148 causes the screw follower 164 to travel along threads of the screw and thereby pivots the needle mount 160 about the pivot joint 150. Pivoting of the needle mount 160 displaces the lower portion of the needle mount 160, and thus the needle 190, primarily in two opposing horizontal directions (X) with respect to the second arm portion 142. Turning of the lead screw 148 in a rotational direction corresponding to a first rotational direction of the motor 146 causes the needle mount 160/needle 190 to move outward in a first direction X, while turning of the lead screw 148 in a rotational direction corresponding to a second rotational direction of the motor 146 causes the needle mount 160/needle 190 to move inward in a second, opposite direction X.

Referring to FIGS. 3A and 3D, the needle mount 160 includes a needle clamp member 166 that is slidably mounted within an open track or slot 168 in the outer surface of the needle mount 160. As shown in FIG. 1, the needle clamp member 166 is configured to securely attach to a head 192 (FIG. 1) of the needle 190. Turning back to FIG. 3A, the clamp member 166 is configured to slide linearly in two opposing directions (V) with respect to the needle mount 160 within the track 168. A needle driving motor 170 is provided in the needle mount 160, and is electrically connected to the electrical connector 161 to receive power from the power source and electrical control signals from the first control element 123 or an external control device connected to port 125 (FIG. 2B). Accordingly, control of operation of the motor 170 using the control element 123 or an external control device is enabled. The motor 170 is configured to rotate in first and second directions through operation of the control element 123, thereby enabling turning of the lead screw 148 in two opposing rotational directions. A lead screw 172 may be connected to the motor 170 such that the lead screw 172 turns in response to rotation of the motor 170. The lead screw 172 may engage a screw follower 167 (FIG. 3A) of the needle clamp member 166 such that turning of the lead screw 172 causes the clamp member 166, and thus the needle 190, to move along the track 168 in the directions V. Turning of the lead screw 172 in a rotational direction corresponding to a first rotational direction of the motor 170 causes the needle clamp member 166/needle 190 to advance downward in a first direction V, while turning of the lead screw 148 in a rotational direction corresponding to a second rotational direction of the motor 170 causes the needle clamp member 166/needle 190 to retract upward in a second, opposite direction V.

While the embodiment shown in FIG. 3A employs lead screws connected to the motors 146, 170, it should be understood that different components, configurations and motor-driven linkages are possible. Additionally, the motors 146, 170 may be replaced by a single motor controllable by the control elements 123, 124 to move the needle mount 160 and advance/retract the needle 190 through selective engagement with separate linkages connecting the motor to the needle mount 160 and the needle clamp member 166.

Still referencing FIGS. 3A and 3D, the needle mount 160 includes clamp members 174 (e.g., resilient arm members) configured to secure the needle cartridge 182 to the needle mount 160. The clamp members 174 may be configured such that the needle cartridge 182 may be pressed within the clamp members 174 and retained by a press fit or snap-in fit (shown in FIG. 1).

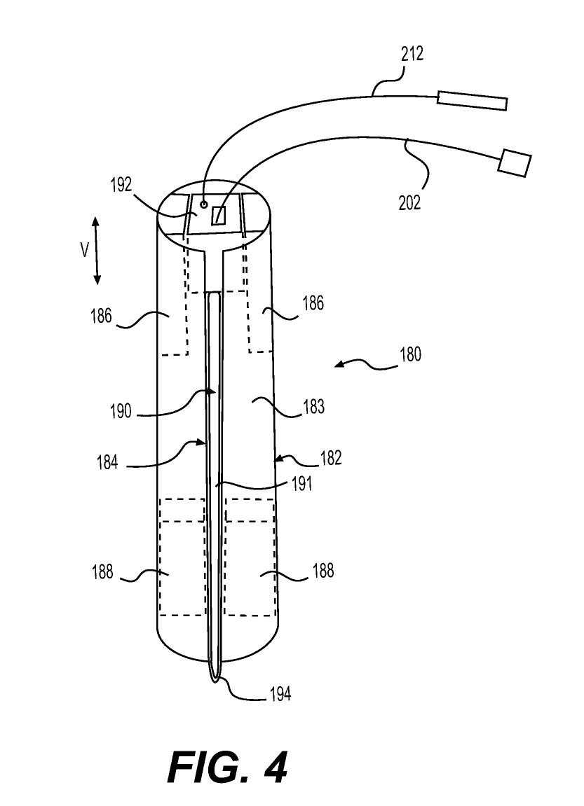

FIG. 4 shows the needle assembly 180 in detail. As shown in FIG. 4, when device 100 is assembled for use, the needle 190 is disposed within the cartridge 182. The cartridge 182 may include a cartridge body 183 that is substantially tubular in shape and includes an elongate needle clamp opening or slot 184 which faces the needle mount 160 when the cartridge 182 is attached to the needle mount 160. The needle clamp opening 184 accommodates the needle clamp member 166, allowing the needle clamp member 166 to move (V) within the cartridge 182 when caused to move by the motor 170. As indicated above, when the device 100 is assembled for use, the needle clamp member 166 is attached to the head 192 (FIG. 1). Accordingly, the needle moves (V) within the cartridge 182 along with the needle clamp member.

First needle guide members 186 and second guide members 188 are provided on an interior surface of the cartridge body 183. The first needle guide members 186 are configured to engage the head 192 of the needle 190, while the second needle guide members are configured to engage the shaft 191 of the needle. The guide members 186, 188 limit motion of the needle in directions transverse to the direction of insertion to promote efficient and stable movement of the needle 190. Particularly, as the tip 194 and shaft 191 of the needle 190 are advanced into the tissue T, the guide members 186, 188 limit deflection and bending of the needle 190 due to resistive forces acting on the tip 194 and the shaft 191. The guide members 186, 188 may be constructed of any material having a desired combination of stiffness and resiliency.

An exemplary nerve block procedure using the system 1 will now be described. As can be understood from FIGS. 1, 2A, 2E and 7, the main body member 110 of the needle positioning device 100 may be attached to the ultrasound probe 20 by placing the probe 20 between the first and second body portions 112, 114 and securing the body portions 112, 114 around the probe 20 by locking the closure mechanism 116. The arm member 130 may be attached to the main body member 110 by inserting the connector 134 into a respective receptacle 127, 129 (FIGS. 2A-2D) of one of the connection ports 126, 128. For an in-plane treatment procedure, the connector 134 is inserted into the receptacle 127. Alternatively, for an out-of-plane treatment procedure, the connector 134 is inserted into one of the receptacles 129. As shown in FIG. 1, the needle cartridge 180, including the needle 190 connected to the syringe 200 and the nerve stimulation device 210, may be attached to the needle mount 160, which is attached to the arm member 130 as described above. The device may be connected to a power supply and may be powered on by operating the power button 120 (FIG. 2B).

As shown in FIGS. 1 and 7, once the system is set up as described above, the ultrasound device 10 may be operated such that the ultrasound probe 20 casts the ultrasound beam B. The user/treatment provider may manipulate the probe 20 over the tissue T such that the user is able to visually identify the target nerve T which is desired to be treated with the local anesthetic medication in order to effectuate the nerve block. The nerve stimulation device 210 may be operated to stimulate the target nerve T and verify its location within the ultrasound beam B. Once the target nerve T is located and the ultrasound beam B is placed in a desired orientation for maintaining visual identification of the target nerve T, the user pivots the second arm portion 142 with respect to the first arm portion 132 to place the needle 190 in a desired position relative to the desired point of entry into the tissue T. Upon placing the needle 190 in the desired position, the arm member 130/second arm portion 142 may be locked into position by operating the pivot lock/release actuator 140.

With the needle 190 secured in the desired position, the user may operate the control element 123 (FIG. 2B) to advance the needle 190 into the tissue T. As the needle 190 is advancing into the tissue T, the user may operate the control element 124 (FIG. 2B) to adjust the insertion angle at which the needle 190 is inserted into the tissue T. Additionally, the control element 124 may be operated to adjust the insertion angle of the needle 190 prior to advancing the needle into the tissue T. The user may continue to advance the needle 190 into the tissue and, if needed, adjust the insertion angle using the control elements 123, 124 until the needle 190 reaches a desired location in the vicinity of the target nerve N or pierces the sheath S of the target nerve N. Thereafter, the user, may operate the syringe 200 to inject the local anesthetic medication into the nerve sheath S and/or surrounding portions of the tissue T.

Once the local anesthetic medication has been administered, the user may operate the control element 123 to retract the needle 190 from the vicinity of the nerve N/nerve sheath S and the tissue T. The user may operate the pivot lock/release actuator 140 to unlock the arm member 130/second arm portion 142, and may then rotate the second arm portion 142 away from the tissue T.

While the above description provides exemplary embodiments related to nerve block treatment procedures, the disclosed devices and methods may be used in other types of treatment procedures and examinations. Furthermore, medications other than local anesthetics may be administered using the disclosed devices, systems and methods. For example, the system 1 may be used for treatment and examination of other material or tissue (e.g., blood vessel cannulation and various organ/tissue injections and biopsies). In such embodiments, the ultrasound device may be used to visually identify a target portion of tissue/material (e.g., a blood vessel, organ or tissue to be treated), the needle 190 may be positioned relative to a desired point of entry into an area around the target portion of tissue/material, and the needle 190 may be advanced into a vicinity of the target portion of tissue/material. The needle 190 may be used to inject medication into the target portion of material tissue/material or withdraw fluid or a tissue/material sample from the target portion of tissue/material.

Additionally, the devices and systems disclosed herein may be used for positioning a needle and treating/examining material other than tissue of living organisms.

Exemplary embodiments have been described above. Nevertheless, it will be understood that various modifications may be made. For example, suitable results may be achieved if the described techniques are performed in a different order and/or components in a described system or device are combined in a different manner and/or replaced or supplemented by other components or their equivalents. Accordingly, other implementations are within the scope of the following claims.

I claim:

1. A needle positioning device for an ultrasound-guided medical treatment or examination, comprising:
a main body member configured to attach to an ultrasound probe;
an arm member comprising
a first arm portion attached to the main body member, and
a second arm portion that is pivotable with respect to the first arm portion; and
a needle mount attached to the second arm portion, the needle mount being configured to retain a needle and vary an angle of insertion of the needle into a material.

2. The needle positioning device of claim 1, comprising at least one motor configured to:
pivot the needle holder to vary the angle of insertion of the needle; and
move the needle linearly with respect to the needle mount.

3. The needle positioning device of claim 2, wherein the main body member comprises control elements configured to control operation of the at least one motor.

4. The needle positioning device of claim 3 comprising wherein:
the arm member comprises a connector;
the main body member comprises at least one connection port electrically connected to a power source and the control elements; and
the at least one connection port is configured to receive the connector to attach the arm member to the main body member and provide the arm member an electrical connection to the power source and the control elements.

5. The needle positioning device of claim 2, wherein the at least one motor comprises:
a first motor disposed in the second arm portion and configured to pivot the needle holder to vary the angle of insertion of the needle; and
a second motor disposed in the needle mount and configured to move the needle linearly with respect to the needle mount.

6. The needle positioning device of claim 1, wherein the second arm portion is pivotable to position the needle in a desired position relative to a point of insertion of the needle into the material.

7. The needle positioning device of claim 1, comprising a needle cartridge attached to the needle mount, wherein the needle is positioned within the needle cartridge.

8. The needle positioning device of claim 1, wherein the needle cartridge comprises:
a cartridge body; and
guide members disposed within the cartridge body and configured to limit movement of the needle in directions transverse to a direction of insertion of the needle into the material.

9. The needle positioning device of claim 1, wherein the main body member comprises a first body portion and a second body portion pivotally attached to the first body portion, and wherein the first body portion and the second body portion are configured to be fastened together to surround the ultrasound probe.

10. The needle positioning device of claim 1, comprising wherein:
the arm member comprises a connector; and
the main body member comprises a connection port configured to receive the connector to attach the arm member to the main body member.

11. The needle positioning device of claim 1, wherein:
the main body member comprises a first end, a second end opposite the first end, a first side extending between the first end and the second end, and a second side extending between the first end and the second end, opposite the first side;
the arm member comprises a connector;
the main body member comprises a first connection port on the first end and a second connection port on the first side or the second side; and
the first connector is configured to be connected to the first connection port or the second connection port to attach the arm member to the main body member.

12. An ultrasound system for performing an ultrasound-guided medical treatment or examination, comprising:
an ultrasound probe configured to cast an ultrasound beam; and
a needle positioning device comprising
a main body member attached to the ultrasound probe,
an arm member attached to the main body member and adjustable to position a needle in a desired position relative to a point of entry into a material, and
a needle mount attached to the arm member, the needle mount being configured to retain a needle and vary an angle of insertion of the needle into the material.

13. The ultrasound system of claim 12, wherein the needle positioning device comprises at least one motor configured to:
pivot the needle holder to vary the angle of insertion of the needle; and
move the needle linearly with respect to the needle mount.

14. The ultrasound system of claim 13, wherein the main body member comprises control elements configured to control operation of the at least one motor.

15. The ultrasound system of claim 14, wherein:
the arm member comprises a connector;
the main body member comprises at least one connection port electrically connected to a power source and the control elements; and
the at least one connection port is configured to receive the connector to attach the arm member to the main body member and provide the arm member an electrical connection to the power source and the control elements.

16. The ultrasound system of claim 13, wherein the at least one motor comprises:
a first motor disposed in the arm member and configured to pivot the needle holder to vary the angle of insertion of the needle;
a second motor disposed in the needle mount and configured to move the needle linearly with respect to the needle mount.

17. The ultrasound system of claim of claim 12, wherein the arm member comprises:
a first arm portion attached to the main body member, and
a second arm portion connected pivotally connected to the first arm portion, wherein the needle mount is pivotally connected to the second arm portion.

18. The ultrasound system of claim 12, comprising a needle cartridge attached to the needle mount, wherein the needle is positioned within the needle cartridge.

19. The ultrasound system of claim 12, wherein the needle cartridge comprises:
a cartridge body; and
guide members disposed within the cartridge body and configured to limit movement of the needle in directions transverse to a direction of insertion of the needle into the material.

20. The ultrasound system of claim 12, wherein:
the arm member comprises a connector;
the main body member comprises a first connection port and a second connection port; and
the connector is configured to be connected to the first connection port or the second connection port to attach the arm member to the main body member,
wherein the arm member is configured to align the needle for insertion into the material transversely to a plane of the ultrasound beam when the connector is connected to the first connection port, and
wherein the arm member is configured to align the needle for insertion into the material substantially entirely within a plane of to the ultrasound beam when the connector is connected to the second connection port.

21. An ultrasound treatment or examination method, comprising:
operating an ultrasound probe to cast an ultrasound beam through a material;
based on the ultrasound beam, adjusting an arm member of a needle positioning device attached to the ultrasound probe to position a needle in a desired position relative to a point of entry into the material;
adjusting a needle mount attached to the arm member to determine an angle of insertion of the needle into the material; and
inserting the needle into the material.

22. The method of claim 21, wherein:
the arm member comprises a first arm portion and a second arm portion pivotally connected to the first arm portion; and
adjusting the arm member comprises pivoting the second arm portion with respect to the first arm portion.

23. The method of claim 21, comprising operating at least one motor to adjust the needle mount and insert the needle into the material, wherein:
adjusting the needle mount comprises pivoting the needle mount with respect to the arm member; and
inserting the needle into the material comprises moving the needle with respect to the needle mount.

24. The method of claim 21, wherein the arm member extends substantially parallel to a plane of the ultrasound beam, and inserting the needle into the material comprises inserting the needle substantially entirely within the plane of the ultrasound beam.

25. The method of claim 21, wherein the arm member extends substantially perpendicular to a plane of the ultrasound beam, and inserting the needle into the material comprises inserting the needle transversely to the plane of the ultrasound beam.
